(19) United States
(12) Patent Application Publication
(10) Pub. No.: US 2015/0024948 A1
(43) Pub. Date: Jan. 22, 2015

(54) METHOD FOR DETECTING BALANCED CHROMOSOMAL ABERRATIONS IN A GENOME

(71) Applicant: ROCHE DIAGNOSTICS OPERATIONS, INC., INDIANAPOLIS, IN (US)

(72) Inventors: Martin Dugas, Muenster (DE); Vera Grossmann, Muenchen (DE); Claudia Haferlach, Inning (DE); Torsten Haferlach, Inning (DE); Wolfgang Kern, Starnberg (DE); Hans-Ulrich Klein, Muenster (DE); Alexander Kohlmann, Neumarkt i.d. OPf (DE); Susanne Schnittger, Muenchen (DE)

(21) Appl. No.: 14/506,996

(22) Filed: Oct. 6, 2014

Related U.S. Application Data
(63) Continuation of application No. 13/458,608, filed on Apr. 27, 2012, now abandoned.
Application No. 13/458,608 is a continuation of application No. PCT/EP2010/006627, filed on Oct. 29, 2010.

(30) Foreign Application Priority Data
Oct. 30, 2009 (EP) 09013670.6

(51) Int. Cl. C12Q 1/68; G06F 19/22

(52) U.S. Cl. CPC C12Q 1/6874; G06F 19/22; C12Q 1/6883; C12Q 1/6886; C12Q 2600/156
USPC 506/2

(57) ABSTRACT

The present disclosure provides methods and systems for the capture and enrichment of target nucleic acids and analysis of the enriched target nucleic acids for detecting balanced chromosomal aberrations including translocations and inversions. The present disclosure provides for the enrichment of targeted sequences in a format whereby one fusion partner gene on a capturing platform is represented to allow subsequent sequencing of chimeric nucleic acids (i.e., nucleic acid strands that carry information on different DNA regions of a genome). Such a design enables identification of novel fusion partner genes occurring as a result of a chromosomal translocation or inversion.

### PRIORITY CLAIM

This application is a continuation of U.S. application Ser. No. 13/458,608 filed Apr. 27, 2012, which is a continuation of International Application No. PCT/EP2010/006627, filed Oct. 29, 2010, which claims the benefit of European Patent Application No. 09013670.6, filed Oct. 30, 2009, the disclosures of which are hereby incorporated by reference in their entirety.

### SEQUENCE LISTING

The instant application contains a Sequence Listing which has been submitted in ASCII format via EFS-Web and is hereby incorporated by reference in its entirety. Said ASCII copy, created on Apr. 10, 2012, is named SEQUENCE_LISTING_{—}26431 US.txt, and is two thousand one hundred and seven bytes in size.

### FIELD OF THE DISCLOSURE

The present disclosure relates to the field of nucleic acid sequence technology. More specifically, the present disclosure relates to nucleic acid sequence analysis.

### BACKGROUND OF THE DISCLOSURE

Nucleic acid microarray technology, such as DNA microarray technology, has enabled the building of an array of millions of nucleic acid sequences (e.g., DNA sequences) within a very small area such as a microscope slide. Additionally, the availability of reference sequences for the entire genome of hundreds of organisms (deposited within a public database), has enabled the use of such microarrays for performing sequence analysis on nucleic acids (e.g., DNA) isolated from a myriad of organisms.

Nucleic acid or DNA microarray technology has been utilized in many areas of basic research as well as clinical diagnostics, for example in gene expression profiling and biomarker discovery (Haferlach T et al. Blood. 2005 Aug. 15; 106(4):1189-98.), mutation detection, allelic and evolutionary sequence comparison, genome mapping, and drug discovery, among other areas. Some applications search for genetic aberrations and point mutations underlying human diseases across the entire human genome. However, the entire human genome is typically too complex to be studied as a whole. Further, in the case of complex diseases, these searches generally result in a single nucleotide polymorphism (SNP) or set of SNPs associated with diseases and/or disease risk. Identifying such SNPs has proved to be an arduous and frequently fruitless task because resequencing large regions of genomic DNA, generally greater than 100 kilobases (Kb), from affected individuals or tissue samples is required to find a single base change or to identify all sequence variants. Resequencing genomic DNA also is required to characterize patient samples with respect to small insertions and deletions.

Other applications may involve the identification of gains and losses of chromosomal sequences which may also be associated with diseases and disease risks, such as cancer including leukemia (see for example Walter M J et al., Proc Natl Acad Sci USA. 2009 Aug. 4; 106(31):12950-5), lymphoma (see for example Martinez-Climent J A et al., 2003, Blood 101:3109-3117), gastric cancer (see for example Weiss M M et al., 2004, Cell. Oncol. 26:307-317), breast cancer (see for example Callagy G et al., 2005, J. Path. 205: 388-396), and prostate cancer (see for example Paris, P L et al., 2004, Hum. Mol. Gen. 13:1303-1313). As such, microarray technology is a tremendously useful tool for scientific investigators and clinicians in their understanding of diseases and therapeutic regimen efficacy in treating diseases.

### SUMMARY OF THE DISCLOSURE

The present disclosure provides a system and methods for the enrichment and analysis of nucleic acid sequences. According to the present disclosure, the system and methods provide novel methods for capturing specific genomic regions for subsequent sequence analysis in order to detect balanced chromosomal aberrations. In some exemplary embodiments, the present disclosure provides methods especially suited for regions of interest which are too large to be amplified by only one or a few polymerase chain reactions (PCR). In some exemplary embodiments, the present disclosure provides methods especially suited for identifying fusion partner genes such as occurring in cancer genomes. Moreover, the novel methods disclosed herein allow for identifying, thus far, unknown fusion partner genes, for example, by capturing one fusion partner but also sequencing the second fusion partner as a result of such a chimeric sequence. According to the instant disclosure, the system and methods disclosed herein may be applied both for translocations and inversions.

According to exemplary embodiments, the present disclosure provides for the enrichment of targeted sequences in a format by representing one fusion partner gene on a capturing platform and allowing subsequent sequencing of chimeric nucleic acids such as nucleic acid strands that carry information on different DNA regions of a genome. As discovered and disclosed herein, the methods and systems of the instant disclosure surprisingly and unexpectedly allow for the identification of novel fusion partner genes occurring as a result of a chromosomal translocation or inversion.

According to some embodiments of the present disclosure, a method for detecting balanced chromosomal aberrations in a genome is provided. The method comprises the steps of:

(a) exposing fragmented, denatured nucleic acid molecules of said genome to multiple, different oligonucleotide probes located on multiple, different sites of a solid support under hybridizing conditions to capture nucleic acid molecules that specifically hybridize to said probes,
wherein said fragmented, denatured nucleic acid molecules have an average size of about 100 to about 1000 nucleotide residues, preferably about 250 to about 800 nucleotide residues and most preferably about 400 to about 600 nucleotide residues, in particular about 500 nucleotide residues,
wherein said oligonucleotide probes have an average size of about 20 to about 100 nucleotides, preferably about 40 to about 85 nucleotides, more preferred about 45 to about 75 nucleotides, in particular about 55 to about 65 nucleotide residues or about 60 nucleotide residues,
(b) separating unbound and non-specifically hybridized nucleic acids from the captured molecules;
(c) eluting the captured molecules from the solid support,
(d) optionally repeating steps (a) to (c) for at least one further cycle with the eluted captured molecules,
(e) determining the nucleic acid sequence of the captured molecules, in particular by means of performing sequencing by synthesis reactions,
(f) comparing the determined sequence to sequences in a database of the reference genome,
(g) identifying sequences in the determined sequence which only partially match or do not match with sequences of the reference genome,
(h) detecting at least one balanced chromosomal aberration.

According to some specific embodiments of the present disclosure, pre-selected, immobilized nucleic acid probes for capturing target nucleic acid sequences from, for example, a genomic sample by hybridizing the sample to probes on a solid support is disclosed. According to some embodiments, the captured target nucleic acids may be washed and eluted off of the probes. In some cases, the eluted genomic sequences may be more amenable to detailed genetic analysis than a sample that has not been subjected to the methods described herein.

An alternative embodiment of the present disclosure is directed to the solution based capture method comprising probe derived amplicons wherein said probes for amplification are affixed to a solid support. The solid support comprises support-immobilized nucleic acid probes to capture specific nucleic acid sequences from a genomic sample. Probe amplification provides probe amplicons in solution which are hybridized to target sequences. Following hybridization of probe amplicons to target sequences, target nucleic acid sequences present in the sample are enriched by capturing and washing the probes and eluting the hybridized target nucleic acids from the captured probes. The target nucleic acid sequence(s) may be further amplified using, for example, non-specific ligation-mediated PCR (LM-PCR), resulting in an amplified pool of PCR products of reduced complexity compared to the original target sample which is further analysed by sequencing as described above.

Consequently, the present disclosure broadly relates to cost-effective, flexible and rapid methods for reducing nucleic acid sample complexity to enrich for target nucleic acids of interest and to facilitate further processing and the identification of fusion or chimeric genes. Generally, the present disclosure provides methods useful, for example, in searching for genetic variants and mutations, single nucleotide polymorphisms (SNPs), sets of SNPs, genomic insertions and deletions in addition to the identification of balanced chromosomal aberrations.

According to some exemplary embodiments of the instant disclosure, a method for detecting balanced chromosomal aberrations in a genome of an organism is provided. The method comprises the steps of exposing fragmented, denatured nucleic acid molecules of the genome to a plurality of oligonucleotide probes bound to different positions of a solid support. The nucleic acid molecules have an average size of about 100 to about 1000 nucleotide residues and the oligonucleotide probes have an average size of about 20 to about 100 nucleotide residues. The method also includes the step of separating nucleic acid molecules bound to one or more of the oligonucleotide probes from nucleic acid molecules not bound to one or more of the oligonucleotide probes and then eluting the nucleic acid molecules bound to one or more of the oligonucleotide probes from the solid support. Thereafter, the nucleic acid molecules which were eluted in the step of eluting are sequenced, thereby getting a determined sequence for the nucleic acid molecules. Also, the method includes the step of comparing the determined sequence to a database comprising a reference genome sequence and identifying sequences in the determined sequence which only partially match or do not match with sequences of the reference genome, thereby detecting at least one balanced chromosomal aberration.

According to some embodiments, the oligonucleotide probes include a linker for binding to the solid support. In various embodiments, the linker may comprise a chemical linker. In some embodiments, the method may further include the steps of ligating at least one adaptor molecule to at least one end of the nucleic acid molecules prior to step exposing and amplifying the nucleic acid molecules which bound to one or more of the oligonucleotide probes with at least one primer comprising a sequence which specifically hybridizes to the adaptor molecule, whereby the step of amplifying is carried out after the step of eluting. Further, according to some embodiments of the instant disclosure, the solid support is either a nucleic acid microarray or a population of beads.

Other embodiments of the instant disclosure include the method of detecting balanced chromosomal aberrations in a genome. The method includes the steps of providing a solid support comprising a plurality of different oligonucleotide probes bound to different positions of the solid support, wherein the oligonucleotide probes have an average size of about 20 to about 100 nucleotides, and providing a plurality of fragmented and denatured nucleic acid molecules having an average size of about 100 to about 1000 nucleotide residues. The method also includes the step of amplifying the oligonucleotide probes, thereby generating amplification products including a binding moiety and being maintained in solution. Thereafter, the method includes the steps of hybridizing the target nucleic acid molecules to the amplification products in solution under specific hybridizing conditions, thereby generating a plurality of hybridization complexes, and separating the hybridization complexes from nucleic acid molecules not hybridized to the amplification products. Next, according to the method, the hybridized target nucleic acid molecules are separated from the amplification product comprising the hybridization complex and sequenced, whereby a determined sequence for the nucleic acid molecules is obtained. According to the method, the determined sequence is compared to a database comprising a reference genome and sequences in the determined sequence which only partially match or do not match with sequences of the reference genome are determined in order for detecting at least one balanced chromosomal aberration.

According to some embodiments, the binding moiety is a biotin moiety. According to some embodiments, oligonucleotide probes having highly repetitive sequences are not used. Further, in some embodiments, the balanced chromosomal aberrations identified may include translocations or inversions.

### BRIEF DESCRIPTION OF THE DRAWINGS

The features of this disclosure, and the manner of attaining them, will become more apparent and the disclosure itself will be better understood by reference to the following description of embodiments of the disclosure taken in conjunction with the accompanying drawing.

FIG. 1 schematically shows an example of the chromosomal translocation AML (with t(9;11)(p22;q23)) and molecular fusion of MLL-MLLT3. On the molecular level MLL (HGNC: 7132) is fused to MLLT3 (HGNC: 7136) from chromosome 9.

FIG. 2 schematically shows capture of both MLL sequences and chimeric nucleic acids according to Example 2.

Corresponding reference characters indicate corresponding parts throughout the several views. Although the drawings represent embodiments of the present disclosure, the drawings are not necessarily to scale and certain features may be exaggerated in order to better illustrate and explain the present disclosure. The exemplifications set out herein illustrate an exemplary embodiment of the disclosure, in one form, and such exemplifications are not to be construed as limiting the scope of the disclosure in any manner.

### BRIEF DESCRIPTION OF THE SEQUENCE LISTING

SEQ ID NO.: 1 is the nucleotide sequence for an exemplary probe according to the present disclosure.

SEQ ID NO.: 2 is the nucleotide sequence for another exemplary probe according to the present disclosure.

SEQ ID NO.: 3 is the nucleotide sequence for the forward primer NSC-0237.

SEQ ID NO.: 4 is the nucleotide sequence for the reverse primer NSC-0237.

SEQ ID NO.: 5 is the nucleotide sequence for the forward primer NSC-0247.

SEQ ID NO.: 6 is the nucleotide sequence for the reverse primer NSC-0247.

SEQ ID NO.: 7 is the nucleotide sequence for the forward primer NSC-0268.

SEQ ID NO.: 8 is the nucleotide sequence for the reverse primer NSC-0268.

SEQ ID NO.: 9 is the nucleotide sequence for the forward primer NSC-0272.

SEQ ID NO.: 10 is the nucleotide sequence for the reverse primer NSC-0272.

Although the sequence listing represents an embodiment of the present disclosure, the sequence listing is not to be construed as limiting the scope of the disclosure in any manner and may be modified in any manner as consistent with the instant disclosure and as set forth herein.

### DETAILED DESCRIPTION OF THE EMBODIMENTS OF THE DISCLOSURE

The embodiments disclosed herein are not intended to be exhaustive or limit the disclosure to the precise form disclosed in the following detailed description. Rather, the embodiments are chosen and described so that others skilled in the art may utilize their teachings.

The advent of nucleic acid microarray technology, for example DNA microarray technology, makes it possible to build an array of millions of nucleic acid sequences, for example DNA sequences, in a very small area, for example on a microscope slide, as e.g. disclosed in U.S. Pat. Nos. 6,375,903 and 5,143,854. Initially, such arrays were created by spotting pre-synthesized DNA sequences onto slides. However, the construction of maskless array synthesizers (MAS) in which light is used to direct synthesis of the DNA sequences, the light direction being performed using a digital micromirror device (DMD), as described in U.S. Pat. No. 6,375,903, allows for in situ synthesis of oligonucleotide sequences directly on the slide itself.

Using a MAS instrument, the selection of oligonucleotide sequences (or DNA sequences) to be constructed on the microarray is under software control allowing for the creation of individually customized arrays based on the particular needs of an investigator. In general, MAS-based oligonucleotide or DNA microarray synthesis technology allows for parallel synthesis of millions of unique oligonucleotide features in a very small area of a standard microscope slide. The microarrays are generally synthesized by using light to direct which oligonucleotides are synthesized at specific locations on an array, these locations are termed features.

The genome is typically too complex to be studied as a whole, thus techniques should be used to reduce its complexity. To address this problem, one solution is to reduce certain types of abundant sequences from a genomic nucleic acid or DNA sample, exemplary methods of such as process include U.S. Pat. No. 6,013,440, Albert et al. (2007, Nat. Meth., 4:903-5), Okou et al. (2007, Nat. Meth. 4:907-9), Olson M. (2007, Nat. Meth. 4:891-892), Hodges et al. (2007, Nat. Genet. 39:1522-1527), Lovett et al. (1991, Proc. Natl. Acad. Sci. 88:9628-9632, describing a method for genomic selection using bacterial artificial chromosomes), International Patent Application Publication WO 2009/053039 and U.S. Patent Applications 2007/0196843, 2008/0194414, and 2009/0221438.

Microarray technology typically comprises a substrate having inherent variability, such as microarray slides, chips, and the like. Variability can take on many forms, for example variability in background, probe/hybridization kinetics, glass source, and the like.

In cancer genomes various chromosomal aberrations are known (see for example Atlas of Genetics and Cytogenetics in Oncology and Haematology. URL http://AtlasGeneticsOncology.org). For example, in leukemia, balanced chromosomal aberrations like translocations and inversions have been proven to have diagnostic and prognostic relevance. For example, a subset of human acute leukemias with a decidedly unfavorable prognosis possesses a chromosomal translocation involving the “Mixed Lineage Leukemia” (MLL, HRX, AU-1) gene on chromosome segment 11q23. The leukemic cells have been classified as “Acute Lymphoblastic Leukemia” (ALL). However, unlike the majority of childhood ALL, the presence of the MLL translocations often results in an early relapse after chemotherapy. Generally, therapeutic treatment is more successful when tailored to the specific type of cancer, in particular with respect to leukemia. Today, the genetic characterization necessary for optimal treatment of acute myeloid leukemia (AML) requires a combination of different labor-intensive methods such as chromosome banding analysis, sequencing for the detection of molecular mutations, and RT-PCR for the confirmation of characteristic fusion genes. Thus, a need exists for accurate and efficient methods for diagnosis of malignancies like leukemia, and for identifying their subclasses other than conventional assays such as metaphase cytogenetics or fluorescence in situ hybridization (FISH).

As used herein, the term “about” refers to a general error range of +/−10%, in particular +/−5%.

As used herein, the term “balanced chromosomal aberration” refers to chromosomal aberrations without visible gain or loss of genetic material, i.e. it refers to the rearrangement of genes, genomes or chromosomes without visible gain or loss of genetic material, whereas the term “unbalanced chromosomal aberrations” refers to aberrations with visible gain or loss of genetic material, i.e. aberrations with partial deletions or with loss of whole chromosomes. Examples of primary balanced chromosomal aberrations are translocations, in particular reciprocal translocations, and inversions. Balanced chromosomal aberrations usually lead to the formation of abnormal chimeric genes, i.e. genes containing at least two different chromosomal sections. According to the present disclosure, the term “balanced chromosomal aberration” include genetic rearrangements with partial deletions at the breaking points. The term “visible gain or loss of genetic material” in this context means the detection of gain or loss of genetic material by means of visible methods like metaphase cytogenetics or in situ hybridization of interface nuclei, e.g. fluorescence or chromatic in situ hybridization.

A used herein, the terms “chimeric gene” and “fusion gene” are used interchangeably and refer to a balanced chromosomal aberration of a gene or genomic region as explained above.

As used herein, the term “gene” or “gene of an organism” means the genomic or chromosomal DNA sequence containing at least one gene.

As used herein, the term “genetic material”, “genetic sequence”, “genomic material” or “genomic sequence” are used interchangeably and refer to chromosomal DNA.

As used herein, the term “hybridization” is used in reference to the pairing of complementary nucleic acids. Hybridization and the strength of hybridization (e.g., the strength of the association between the nucleic acids) is affected by such factors as the degree of complementary between the nucleic acids, stringency of the conditions involved, the melting temperature (Tₘ) of the formed hybrid, and the G:C ratio of the nucleic acids. While the present disclosure is not limited to a particular set of hybridization conditions, stringent hybridization conditions are generally employed. Stringent hybridization conditions are sequence dependent and differ with varying environmental parameters (e.g., salt concentrations, presence of organics, etc.). Generally, “stringent” conditions are selected to be about 50° C. to about 20° C. lower than the Tₘ for the specific nucleic acid sequence at a defined ionic strength and pH. In some embodiments, stringent conditions may be about 5° C. to 10° C. lower than the thermal melting point for a specific nucleic acid bound to a complementary nucleic acid. The Tₘ is the temperature (under defined ionic strength and pH) at which 50% of a nucleic acid (e.g., target nucleic acid) hybridizes to a perfectly matched probe.

As used herein, the term “isolate” when used in relation to a nucleic acid, as in “isolating a nucleic acid” refers to a nucleic acid sequence that is identified and separated from at least one component or contaminant with which it is ordinarily associated in its natural source. The isolated nucleic acid, oligonucleotide, or polynucleotide may be present in single-stranded or double-stranded form.

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced, (e.g., in the presence of nucleotides and an inducing agent such as DNA polymerase and at a suitable temperature and pH). The primer will generally be single stranded for maximum efficiency in amplification (although double-stranded primers are possible). Also, the primer wil generally be an oligodeoxyribonucleotide. The primer will be sufficiently long to prime the synthesis of extension products in the presence of the inducing agent. The exact lengths of the primers will depend on many factors, including temperature, source of primer and the use of the method.

As used herein, the term “probe” refers to an oligonucleotide whether occurring naturally as in a purified restriction digest or produced synthetically, recombinantly or by PCR amplification, that is capable of hybridizing to at least a portion of another oligonucleotide of interest, for example target nucleic acid sequences. A probe is generally single-stranded. Probes are useful in the detection, identification and isolation of particular gene sequences.

As used herein, the term “sample” is used in its broadest sense. In one sense, it is meant to include a specimen or culture obtained from any source, including a biological source. Biological samples may be obtained from animals (including humans) and encompass fluids, solids, tissues, and gases. Biological samples include blood products, such as plasma, serum and the like. As such, a “sample of nucleic acids” or a “nucleic acid sample”, a “target sample” comprises nucleic acids (e.g., DNA, RNA, cDNA, mRNA, tRNA, miRNA, etc.) from any source. According to the present disclosure, a nucleic acid sample may derive from a biological source, such as a human or non-human cell, tissue, and the like. The term “non-human” refers to all non-human animals and entities including, but are not limited to, vertebrates such as rodents, non-human primates, ovines, bovines, ruminants, lagomorphs, porcines, caprines, equines, canines, felines, ayes, etc. Non-human also includes invertebrates and prokaryotic organisms such as bacteria, plants, yeast, viruses, and the like. As such, a nucleic acid sample used in methods and systems of the present disclosure includes a nucleic acid sample derived from any organism, either eukaryotic or prokaryotic. “Stringent conditions” or “high stringency conditions,” for example, can be hybridization in 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 mg/ml), 0.1% SDS, and 10% dextran sulfate at 42° C., with washes at 42° C. in 0.2% SSC (sodium chloride/sodium citrate) and 50% formamide at 55° C., followed by a wash with 0.1×SSC containing EDTA at 55° C. By way of example, but not limitation, it is contemplated that buffers containing 35% formamide, 5×SSC, and 0.1% (w/v) sodium dodecyl sulfate (SDS) are suitable for hybridizing under moderately non-stringent conditions at 45° C. for 16-72 hours. Furthermore, it is envisioned that the formamide concentration may be suitably adjusted between a range of 20-45% depending on the probe length and the level of stringency desired. Additional examples of hybridization conditions are provided in several laboratory manual known for a person skilled in the art. Similarly, “stringent” wash conditions are ordinarily determined empirically for hybridization of a target to a probe, or a probe derived amplicon. The amplicon/target are hybridized (for example, under stringent hybridization conditions) and then washed with buffers containing successively lower concentrations of salts, or higher concentrations of detergents, or at increasing temperatures until the signal-to-noise ratio for specific to non-specific hybridization is high enough to facilitate detection of specific hybridization. Stringent temperature conditions may include temperatures in excess of about 30° C., more usually in excess of about 37° C., and occasionally in excess of about 45° C. Stringent salt conditions may be less than about 1000 mM, for example less than about 500 mM, or even less than about 150 mM (Wetmur et al., 1966, J. Mol. Biol., 31:349-370; Wetmur, 1991, Critical Reviews in Biochemistry and Molecular Biology, 26:227-259).

As used herein, the term “target nucleic acid molecules” and “target nucleic acid sequences” are used interchangeably and refer to molecules or sequences from a target genomic region to be studied. The pre-selected probes determine the range of targeted nucleic acid molecules. Thus, the “target” is sought to be sorted out from other nucleic acid sequences. A “segment” is defined as a region of nucleic acid within the target sequence, as is a “fragment” or a “portion” of a nucleic acid sequence.

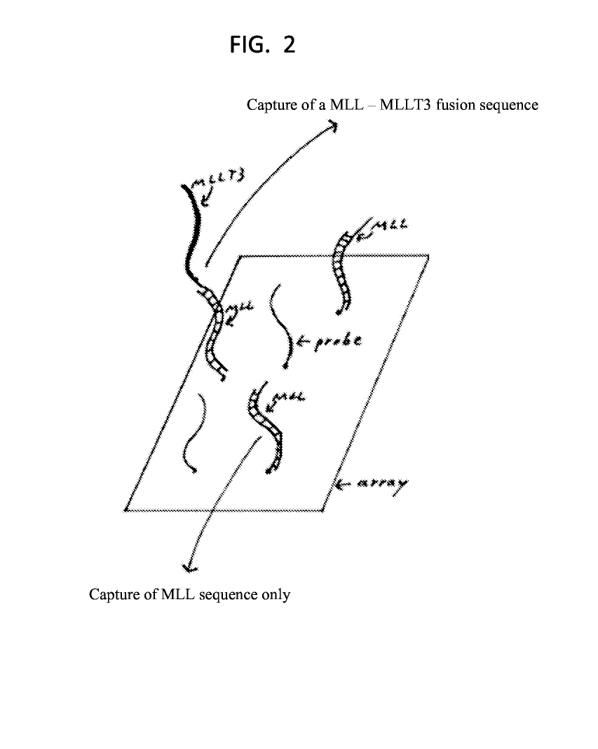

As noted above, the present disclosure concerns exposing fragmented, denatured nucleic acid molecules of a genome to multiple, different oligonucleotide probes located on multiple, different sites of a solid support under hybridizing conditions to capture nucleic acid molecules that specifically hybridize to said probes (see FIG. 2).

Specifically referring to FIG. 2, chip probes are presented, the probes being designed to capture nucleic acids of a gene of interest (for example, MLL on chromosome 11, q23). The MLL gene sequences hybridize to the capture probes, but as a result of a translocation, other sequences also hybridize thereto. As such, this nucleic acid molecule has chimeric properties (i.e., it contains gene information for a first gene, here MLL, and from a second gene, the molecular fusion partner gene, here MLLT3).

According to some embodiments, the fragmented, denatured nucleic acid molecules may have an average size of about 100 to about 1000 nucleotide residues, for example about 250 to about 800 nucleotide residues or about 400 to about 600 nucleotide residues. In come embodiments, the average size may be about 500 nucleotide residues. The nucleic acid fragments of random- or non-random size may be produced by methods known to a person skilled in the art, such as by chemical, physical or enzymatic fragmentation. Chemical fragmentation can employ metal ions and complexes (e.g., Fe-EDTA). Physical methods may include sonication, hydrodynamic shearing or nebulization (see e.g., European patent application EP 0 552 290). Enzymatic protocols may employ nucleases such as micrococcal nuclease (Mnase) or exo-nucleases (such as Exol or Bal31) or restriction endonucleases. According to exemplary embodiments, the genomic DNA may be fragmented by mechanical stress such as sonication. As noted above, the average size of the DNA fragments is generally small (≦1000 bp) and depends on the sequencing method to be applied.

In general, the nucleic acid molecules to be fragmented are genomic DNA molecules, in some cases containing the whole genome or at least one gene or chromosome of an organism, or at least one genomic nucleic acid molecule with a size of about 50 kb or more, at least about 200 kb, at least about 500 kb, at least about 1 Mb, at least about 2 Mb or at least about 5 Mb, a size between about 100 kb and about 5 Mb, between about 200 kb and about 5 Mb, between about 500 kb and about 5 Mb, between about 1 Mb and about 2 Mb or between 2 Mb and about 5 Mb.

As noted above, denaturation of the nucleic acid fragments to a single-stranded state can be carried out using, for example, a chemical or thermal denaturing process.

The oligonucleotide probes, in general, have an average size of about 20 to about 100 nucleotides, although these sizes may vary. Exemplary embodiments include probes having a size of about 40 to about 85 nucleotides, about 45 to about 75 nucleotides, about 55 to about 65 nucleotide residues, and even about 60 nucleotide residues. The probes may define a plurality of exons, introns or regulatory sequences from a plurality of genetic loci because fusion breakpoints can occur both in introns and exons of genes. Therefore, probes are designed to cover as much as possible contiguous region of a gene of interest. Such multiple probes may define the complete sequence of at least one single genetic locus of an organism, said locus having a size of at least about 50 kb, at least 100 kb, least 1 Mb or any size as specified herein, or from at least one gene or at least one chromosome of an organism, from at least about 90%, at least about 95%, at least about 98% of the gene or genome of an organism, such as a human gene or genome. Said multiple probes can define sites known to contain a balanced chromosomal aberration. The multiple probes can also define a tiling array. Such a tiling array in the context of the present disclosure is defined as being designed to capture the complete sequence of at least one complete chromosome. In this context, the term “define” is understood in such a way that the population of multiple probes comprises at least one probe for each target sequence that shall become enriched. For example, according to some embodiments, the population of multiple probes may additionally comprises at least a second probe for each target sequence that shall become enriched, characterized in that said second probe has a sequence which is complementary to said first sequence.

As an example, the genomic region can be a genomic region representing a gene known to be involved in balanced chromosomal aberrations. Breakpoints, in such regions, may occur both in introns and exons, therefore probes may represent the genomic region as complete as possible. Alternatively, to increase the likelihood that desired non-unique or difficult-to-capture targets are enriched, the probes can be directed to sequences associated with (e.g., on the same fragment as, but separate from) the actual target sequence, in which case genomic fragments containing both the desired target and associated sequences will be captured and enriched. The associated sequences can be adjacent or spaced apart from the target sequences, but the skilled person will appreciate that the closer the two portions are to one another, the more likely it will be that genomic fragments will contain both portions. According to embodiments of the present disclosure, the captured sequences differ from a contiguous genomic region, wherein, desired target sequences capture a specific nucleic acid molecule. In the case of a balanced chromosomal aberration the captured nucleic acid molecule will contain sequences mapping to the corresponding capture probe and may also include sequences derived from a fusion partner (which may be derived from a different part of the genome). As an example, these sequences may be from a different chromosome, for example as a result of a translocation (e.g., t(9;11)(p22;q23)). Additionally, these sequences may also be from the same chromosome (e.g., as a result of an inversion such as inv(16)(p13q22)). Thus, the system and methods disclosed herein, aide in the identification of hitherto unknown fusion partner genes.

In some embodiments, to further reduce the limited impact of cross-hybridization by off-target molecules, thereby enhancing the integrity of the enrichment, sequential rounds of capture using distinct but related capture probe sets directed to the target region can be performed. Related probes, for example, are probes corresponding to regions in close proximity to one another in the genome that can, therefore, hybridize to the same genomic DNA fragment.

These probes may be either designed to be overlapping probes, meaning that the starting nucleotides of adjacent probes are less than the length of a probe, or non-overlapping probes, where the distance between adjacent probes are greater than the length of a probe. The distance between adjacent probes is generally overlapping, with spacing between the starting nucleotide of two probes varying between 1 and 100 bases. This distance can be varied to cause some genomic regions to be targeted by a larger number of probes than others. This variation can be used to modulate the capture efficiency of individual genomic regions, normalizing capture. Probes may also be tested for uniqueness in the genome.

To avoid non-specific binding of genomic elements to capture arrays, highly repetitive elements of the genome should be excluded from selection microarray designs. In some embodiments, the oligonucleotide probes do not contain highly repetitive sequences to reduce the likelihood of non-specific binding between the microarrays and genomic nucleic acid molecules. For example, in some embodiments, the average 15-mer frequency of each probe may be calculated by comparing the frequencies of all 15-mers present in the probe against a pre-computed frequency histogram of all possible 15-mer probes in the human genome. The likelihood that the probe represents a repetitive region of the genome increases as the average 15-mer frequency increases. According to some embodiments, only probes having an average 15-mer frequency below about 100 are included on the solid support. Repetitive DNA sequences can also be depleted using a subtraction hybridization protocol as describe by Craig et al. (1997) Hum. Genet., 100: 472-476).

The solid support according to the present disclosure is usually a slide, chip or bead, for example a nucleic acid microarray or a population of beads. Said support may be glass, metal, ceramic and/or polymeric, or the like. General immobilization methods include spotting, photolithography or in situ synthesis. In case said solid support is a chip or microarray, it is possible to synthesize the oligonucleotide capture probes in situ directly onto said solid support. For example, the probes may be synthesized on the microarray using a maskless array synthesizer (see e.g. U.S. Pat. No. 6,375,903, for example). The lengths of the multiple oligonucleotide probes may vary (as explained above). The probes can be designed for convenient release from the solid support. For example, at or near the support-proximal probe termini, an acid- or alkali-labile nucleic acid sequence that releases the probes under conditions of low or high pH, respectively, may be provided (using any of various cleavable linker chemistries known in the art). According to some embodiments, the support may be provided by a column having a fluid inlet and outlet. The art is familiar with methods for immobilizing nucleic acids onto supports, for example by incorporating a biotinylated nucleotide into the probes and coating the support with streptavidin such that the coated support non-covalently attracts and immobilizes the probes in the pool. The length of the linker can range between about 12 and about 100 base pairs, including a range between about 18 and 100 base pairs, and between about 20 and 24 base pairs.

In embodiments in which the solid support is a population of beads, the capture probes may be initially synthesized on a microarray using a maskless array synthesizer, for example, then released or cleaved off according to known standard methods, optionally amplified and then immobilized on said population of beads according to methods known in the art. The beads may be packed into a column so that a sample is loaded and passed through the column for reducing genetic complexity. Alternatively, in order to improve the hybridization kinetics, hybridization may take place in an aqueous solution comprising the beads with the immobilized multiple oligonucleotide molecules in suspension.

In one embodiment, the multiple different oligonucleotide probes may each carry a chemical group or linker (i.e. a moiety which allows for immobilization onto a solid support), also named an immobilizable group (see dots on the array in FIG. 2). Then the step of exposing the fragmented, denatured nucleic acid molecules of the sample to the multiple, different oligonucleotide probes, under hybridizing conditions, is performed in an aqueous solution and immobilization onto an appropriate solid support takes place subsequently. For example, such a moiety may be biotin which can be used for immobilization on a streptavidin coated solid support. In another embodiment, such a moiety may be a hapten like digoxygenin, which can be used for immobilization on a solid support coated with a hapten recognizing antibody (e.g., a digoxygenin binding antibody).

In a specific embodiment, the plurality of immobilized probes is characterized by normalized capture performance. A goal of such normalization is to deliver one gene per read. For example, the number of sequencing reactions required to effectively analyze each target region can be reduced by normalizing the number of copies of each target sequence in the enriched population such that across the set of probes the capture performance of distinct probes are normalized, on the basis of a combination of fitness and other probe attributes. Fitness, characterized by a “capture metric,” can be ascertained either informatically or empirically. In one approach, the ability of the target molecules to bind can be adjusted by providing so-called isothermal (Tm-balanced) oligonucleotide probes, as described in U.S. Published Patent Application No. US2005/0282209, that enable uniform probe performance, eliminate hybridization artifacts and/or bias and provide higher quality output. Probe lengths are adjusted as specified above to equalize the melting temperature (e.g. Tm=76° C., typically about 55° C. to about 76° C., in particular about 72° C. to about 76° C.) across the entire set. Thus, probes are optimized to perform equivalently at a given stringency in the genomic regions of interest, including AT- and GC-rich regions. Related, the sequence of individual probes can be adjusted, using natural bases or synthetic base analogs such as inositol, or a combination thereof to achieve a desired capture fitness of those probes. Similarly, locked nucleic acid probes, peptide nucleic acid probes or the like having structures that yield desired capture performance can be employed. The skilled artisan in possession of this disclosure will appreciate that probe length, melting temperature and sequence can be coordinately adjusted for any given probe to arrive at a desired capture performance for the probe. Conveniently, the melting temperature (Tm) of the probe can be calculated using the formula: Tm=5×(Gn+Cn)+1×(An+Tn), where n is the number of each specific base (A, T, G or C) present on the probe.

Capture performance can also be normalized by ascertaining the capture fitness of probes in the probe set, and then adjusting the quantity of individual probes on the solid support accordingly. For example, if a first probe captures twenty times as much nucleic acid as a second probe, then the capture performance of both probes can be equalized by providing twenty times as many copies of the second probe, for example by increasing by twenty-fold the number of features displaying the second probe. If the probes are prepared serially and applied to the solid support, the concentration of individual probes in the pool can be varied in the same way.

Still further, another strategy for normalizing capture of target nucleic acids is to subject the eluted target molecules to a second round of hybridization against the probes under less stringent conditions than were used for the first hybridization round. Apart from the substantial enrichment in the first hybridization that reduces complexity relative to the original genomic nucleic acid, the second hybridization can be conducted under hybridization conditions that saturate all capture probes. Presuming that substantially equal amounts of the capture probes are provided on the solid support, saturation of the probes will ensure that substantially equal amounts of each target are eluted after the second hybridization and washing.

Another normalizing strategy follows the elution and amplification of captured target molecules from the solid support. Target molecules in the eluate are denatured using, for example, a chemical or thermal denaturing process, to a single-stranded state and are re-annealed. Kinetic considerations dictate that abundant species re-anneal before less abundant species. As such, by removing the initial fraction of re-annealed species, the remaining single-stranded species may be balanced relative to the initial population in the eluate. The timing required for optimal removal of abundant species may be determined empirically.

The normalized capture performance may be achieved by methods as described above, typically comprising the steps of a) ascertaining the capture fitness of probes in the probe set; and b) adjusting the quantity of at least one probe on the solid support. Alternatively, the normalized capture performance may be achieved by a method comprising the steps of a) ascertaining the capture fitness of probes in the probe set; and b) adjusting at least one of the sequence, the melting temperature and the probe length of at least one probe on the solid support. Still alternatively, the normalized capture performance may be achieved by a method comprising the steps of a) exposing the captured molecules to the at least one immobilized probe on the solid support under less stringent conditions than in the first exposing step such that the at least one probe is saturated, b) washing unbound and non-specifically bound nucleic acids from the solid support; and c) eluting the bound target nucleic acids from the solid support. Still alternatively, the normalized capture performance may be achieved by a method comprising the steps of a) denaturing the eluted captured molecules to a single-stranded state; b) re-annealing the single-stranded molecules until a portion of the molecules are double-stranded; and discarding the double-stranded molecules and c) retaining the single-stranded molecules.

Usually at least one immobilized probe hybridizes to a genomic region of interest on nucleic acid fragments in the sample. Alternatively, the at least one immobilized oligonucleotide probe may hybridize to sequences on target nucleic acid fragments comprising a genomic region of interest, the hybridizing sequences being separate from the genomic region of interest. Furthermore, it is also within the scope of the present disclosure, that at least a second hybridization step using at least one oligonucleotide probe related to but distinct from the at least one probe used in the initial hybridization is performed.

Advantageously, the method of the present disclosure further comprises the step of ligating adaptor molecules to one or both ends of the fragmented nucleic acid molecules. According to some embodiments, adaptor molecules are ligated to both ends of the fragmented nucleic acid molecules. Adaptor molecules in the context of the present disclosure may be defined as blunt ended double stranded oligonucleotides. In addition, the inventive method may further comprise the step of amplification of said nucleic acid molecules with at least one primer, said primer comprising a sequence which corresponds to or specifically hybridizes with the sequence of said adaptor molecules.

According to some embodiments, a double stranded target molecule itself may be blunt ended, which may aide in the ligation of adapter molecules thereto. For example, the double stranded target molecules may be subjected to a fill-in reaction with a DNA polymerase such as T4-DNA polymerase or Klenow polymerase in the presence of desoxynucleoside triphposphates, which results in blunt ended target molecules. T4 polynucleotide kinase may be added prior to the ligation in order to add phosphate groups to the 5′ terminus for the subsequent ligation step. Subsequent ligation of the adaptors (short double stranded blunt end DNA oligonucleotides with about 3-20 base pairs) onto the polished target DNA may be performed according to any method which is known in the art, for example by means of a T4-DNA ligase reaction.

The ligation may be performed prior to or after the step of exposing a sample that comprises fragmented, denatured nucleic acid molecules to multiple, different oligonucleotide probes under hybridizing conditions to capture target nucleic acid molecules that hybridize to said probes. In case ligation is performed subsequently, the enriched nucleic acids which are released from the solid support in single stranded form should be re-annealed first followed by a primer extension reaction and a fill-in reaction according to standard methods known in the art.

Ligation of said adaptor molecules allows for a step of subsequent amplification of the captured molecules. Independent from whether ligation takes place prior to or after the capturing step. Alternative embodiments also exist. For example, according to an alternate embodiment of the present disclosure, one type of adaptor molecules is used. This results in population of fragments with identical terminal sequences at both ends of the fragment. As a consequence, it is sufficient to use only one primer in a potential subsequent amplification step. In another alternative embodiment, two types of adaptor molecules A and B are used. This results in a population of enriched molecules composed of three different types: (i) fragments having one adaptor (A) at one end and another adaptor (B) at the other end, (ii) fragments having adaptors A at both ends, and (iii) fragments having adaptors B at both ends.

Amplification and sequencing of enriched molecules (according to type (i, above), may be performed with the 454 Life Sciences (USA) GS20 and GSFLX instrument (see e.g. GS20 Library Prep Manual, December 2006; WO 2004/070007). If one of said adaptors (for example, adaptor B) carries a biotin modification, molecules ii (above) and iii (above) may be bound on streptavidin (SA) coated magnetic particles for further isolation and the products of ii may be washed away. In cases in which the enriched and SA-immobilized DNA is single stranded following elution from the capture array/solid support, it may be advantageous to make the DNA double-stranded. In this case primers complementary to adaptor A may be added to the washed SA pull down products. Since moieties that are B-B (iii above) do not have A or its complement available, only A-B adapted and SA captured products will be made double stranded following primer-extension from an A complement primer. Subsequently, the double stranded DNA molecules that have been bound to said magnetic particles are thermally or chemically (e.g., with NaOH) denatured in such a way that the newly synthesized strand is released into solution. Due to the tight biotin/streptavidin bonding, for example, molecules with only two adaptors B will not be released into solution. The only strand available for release is the A-complement to B-complement primer-extension synthesized strand. Said solution comprising single stranded target molecules with an adaptor A at one end and an adaptor B at the other end may subsequently be bound on a further type of beads comprising a capture sequence which is sufficiently complementary to the adaptor A or B sequences for further processing.

The second general step (b) concerns the separation of unbound and non-specifically hybridized nucleic acids from the captured molecules. In some embodiments, the separation can be carried out with means of biotin attached to the captured target nucleic acid molecules. In this case the capture substrate, such as a bead for example a paramagnetic particle, is coated with streptavidin for separation of the target nucleic acid molecule from non-specifically hybridized target nucleic acid molecules. In some embodiments, the captured target nucleic acid molecules are washed prior to elution of the bound or captured molecules.

The next general step (c) concerns the elution of the captured molecules form the solid support, e.g. by an alkaline solution, for example in an eluate pool which has now a reduced genetic complexity relative to the original sample.

Steps (a) to (c) as well as the intermediate steps as described above can be repeated for at least one further cycle with the eluted captured molecules.

The next general step (e) concerns the determination of the nucleic acid sequence of the captured molecules. Sequencing can be performed by a number of different methods, such as array-based-, shotgun-, capillary-, or other sequencing methods known to the art, for example by employing sequencing by synthesis technology. Sequencing by synthesis according to the prior art is defined as any sequencing method which monitors the generation of side products upon incorporation of a specific deoxynucleoside-triphosphate during the sequencing reaction (Hyman, 1988, Anal. Biochem. 174:423-436; Rhonaghi et al., 1998, Science 281:363-365).

One particular embodiment of the sequencing by synthesis reaction is the pyrophosphate sequencing method. In this case, generation of pyrophosphate during nucleotide incorporation is monitored by means of an enzymatic cascade which finally results in the generation of a chemo-luminescent signal. For example, the 454 Genome Sequencer System (Roche Applied Science Cat. No. 04 760 085 001) is based on the pyrophosphate sequencing technology. Other suitable DNA sequencers are the Genome Analyzer IIx (illumina Inc., San Diego) and the SOLiD™ System (applied biosystems). For sequencing on a 454 GS20 or 454 FLX instrument, the average genomic DNA fragment size should be in the range of 200 or 600 bp, respectively. Alternatively, the sequencing by synthesis reaction may be a terminator dye type sequencing reaction. In such a case, the incorporated dNTP building blocks comprise a detectable label, which may be a fluorescent label that prevents further extension of the nascent DNA strand. The label is then removed and detected upon incorporation of the dNTP building block into the template/primer extension hybrid for example by means of using a DNA polymerase comprising a 3′-5′ exonuclease or proofreading activity.

In case of the Genome Sequencer workflow (Roche Applied Science Catalog No. 04 896 548 001), in a first step, (clonal) amplification is performed by emulsion PCR. Thus, it is also within the scope of the present disclosure, that the step of amplification is performed by emulsion PCR methods. The beads carrying the clonally amplified target nucleic acids may then become arbitrarily transferred into a picotiter plate according to the manufacturer's protocol and subjected to a pyrophosphate sequencing reaction for sequence determination.

The next general step (f) concerns the comparison of the determined sequence to sequences in a database of the reference genome. Such database may contain the whole genome or at least one chromosome of an organism, or at least about 90% of the at least one chromosome of an organism, in particular a human genome or chromosome. In some embodiments, any primer or adaptor sequence may be removed in silico prior to this step (i.e., with the help of a suitable computer program such as gsMapper Version 2.0.01 from Life Sciences, USA).

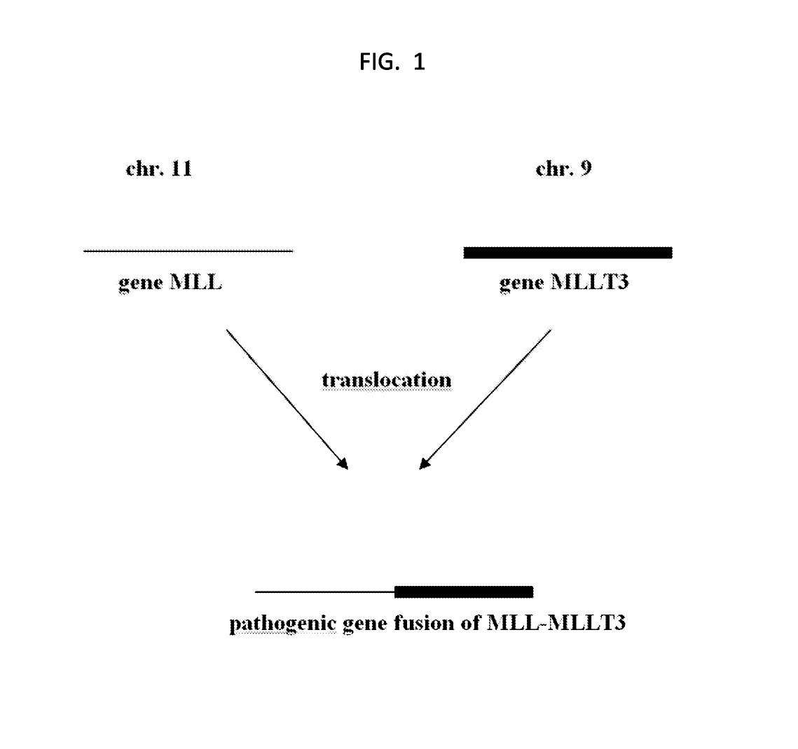

The next general steps (g) and (h) concern the identification of sequences which only partially match or do not match with sequences of the reference genome. These so-called “unmapped” or only “partially mapped” sequences are of particular interest, since these sequences may contain information on distinct fusion genes as a result of a chromosomal aberration. Generally, as a result of a translocation or inversion, fusion or chimeric genes occur (see FIG. 1). According to methods of the present disclosure, it is sufficient to represent only one fusion partner on the capturing assay since nucleic sequences from the second fusion partner will also be captured by probes of the other fusion partner (see FIG. 2). As such, this method will not only allow detecting known fusion events, but also identify novel fusion partner genes. This is a particular advantage of the present disclosure because neither cytogenetic nor molecular genetic analyses are able to fully identify or detect balanced chromosomal aberrations or to fully resolve a molecular fusion gene. Moreover, also reciprocal fusion genes can be detected by the present disclosure. Such sequences are generally identified with the help of a suitable computer program. Consequently, at least one balanced chromosomal aberration can easily be detected in a one-step approach.

An alternative method according to the instant disclosure for detecting balanced chromosomal aberrations in a genome is a method comprising the steps of exposing fragmented, denatured nucleic acid molecules of a target population to multiple, different oligonucleotide probe derived amplicons wherein the amplicons are in solution and wherein the amplicons further comprise a binding moiety, under hybridizing conditions to capture nucleic acid molecules that specifically hybridize to the probe amplicons, binding or capturing the complexes of hybridized molecules by binding the binding moiety found on the probe amplicon to its binding partner (e.g., biotin/SA, digoxigenin/anti-digoxigenin, 6HIS/nickel, etc.), separating unbound and non-specifically hybridized nucleic acids from the bound probe amplicons, eluting the hybridized target molecules from the amplicons, and sequencing the target molecules. In some embodiments, such a method may comprise the following steps:

(a) providing:
i) a solid support comprising multiple, different oligonucleotide probes located on multiple, different sites of the solid support, wherein said oligonucleotide probes have an average size of about 20 to about 100 nucleotides, preferably about 40 to about 85 nucleotides, more preferred about 45 to about 75 nucleotides, in particular about 55 to about 65 nucleotide residues or about 60 nucleotide residues,
ii) a nucleic acid sample comprising target nucleic acid molecules,
   (b) amplifying said oligonucleotide probes wherein the amplification products comprise a binding moiety and wherein said amplification products are maintained in solution,
   (c) hybridizing the target nucleic acid molecules to said amplification products in solution under specific hybridizing conditions, wherein, prior to hybridization, the target nucleic acid molecules are fragmented and denatured and have an average size of about 100 to about 1000 nucleotide residues, preferably about 250 to about 800 nucleotide residues and most preferably about 400 to about 600 nucleotide residues, in particular about 500 nucleotide residues,
   (d) separating the hybridization complexes of target nucleic acid molecules and amplification products from non-specifically hybridized nucleic acids by said binding moiety,
   (e) separating the target nucleic acid molecules from the complex,
   (f) determining the nucleic acid sequence of the separated target nucleic acid molecules, in particular by means of performing sequencing by synthesis reactions,
   (g) comparing the determined sequence to sequences in a database of the reference genome,
   (h) identifying sequences in the determined sequence which only partially match or do not match with sequences of the reference genome, and
   (i) detecting at least one balanced chromosomal aberration.

According to such methods, the target nucleic acid molecules are hybridized with oligonucleotide probes containing a binding moiety in solution (in order to reduce the complexity of the target nucleic acids molecules as described in WO2009/053039). Steps (f) to (i) of the exemplified method are the same as steps (e) to (h) of the embodiment of the present disclosure described above. Therefore, the above-specified features also apply for the alternative method outlined above.

In some embodiments, the multiple, different oligonucleotide probes each contain a chemical group or linker being able to bind to a solid support, as described above. Furthermore, the fragmented target nucleic acid molecules may further comprise adaptor molecules at one or both ends, as described above. In addition, the oligonucleotide probes may further comprise primer binding sequences at one or both ends of said probes, whereas when present at both ends of the probes the primer binding sequences may be the same or be different, as also described above. The length of the linker may range between about 12 and about 100 base pairs, including a range between about 18 and 100 base pairs, and between about 20 and 24 base pairs. Adaptor molecules in the context of the present disclosure may also be defined as blunt-ended double-stranded oligonucleotides.

Generally, the amplification reaction comprises polymerase chain reaction, for example exponential polymerase chain reaction and/or asymmetric polymerase chain reaction.

According to another embodiment, the binding moiety may be a biotin binding moiety, wherein said separating comprises binding said biotin binding moiety to a streptavidin coated substrate, for example to a straptavidin coated paramagnetic particle.

A solution comprising the probe derived amplicons may be transferred to, for example, a tube, well, or other vessel and maintained in solution. It is contemplated that one or more additional rounds of amplification to boost the production of the amplicon strand that comprises the binding moiety, for example by asymmetric PCR, may additionally be performed. A nucleic acid sample, fragmented and denatured to yield fragmented single stranded target sequences, is added to the amplicons in solution and hybridization is allowed to occur between the probe derived amplicons and the fragmented single stranded target nucleic acid sample. After hybridization, nucleic acids that do not hybridize, or that hybridize non-specifically, are separated from the amplicon/target complex by capturing the amplicon/target complex via the binding moiety and washing the amplicon/target complex. For example, if the binding moiety is biotin, a streptavidin coated substrate is used to capture the complex. The bound complex is washed, for example with one or more washing solutions. The remaining nucleic acids (e.g., specifically bound to the amplicons) are eluted from the complex, for example, by using water or an elution buffer (e.g., comprising TRIS buffer and/or EDTA) to yield an eluate enriched for the target nucleic acid sequences.

Therefore, the present method further comprises washing said hybridization complexes prior to separating the target nucleic acid molecules from the complex. The method may also further comprise the step of amplification the separated target nucleic acid molecule prior to step (f), by emulsion polymerase chain reaction for example.

As also described above, the nucleic acid molecules are generally genomic DNA molecules, for example containing the whole genome, at least one gene of an organism or at least one chromosome of an organism, or at least one genomic nucleic acid molecule with a size of at least about 50 kb, at least about 200 kb, at least about 500 kb, at least about 1 Mb, at least about 2 Mb or at least about 5 Mb.

As also described above, the oligonucleotide probes contain exons, introns and/or regulatory sequences from at least a part of a genome of an organism, having a size of at least about 50 kb, at least about 100 kb, at least about 1 Mb, or at least one of the sizes as specified above, or from at least one gene or at least one chromosome of an organism, at least about 90%, at least about 95%, or at least about 98% of the gene or genome of an organism, in particular a human gene or genome.

As noted herein, probes with highly repetitive sequences may also be excluded as described above and the database of the reference genome may contain the whole genome or at least one chromosome of an organism, or at least about 90%, in particular at least about 95%, at least about 98% of the genome or of at least one chromosome of an organism, such as a human genome or chromosome.

According to embodiments of the instant disclosure, any primer or adaptor sequence may be removed in silico prior to step (f) and the solid support may comprise either a nucleic acid microarray or a population of beads. Other solid supports are also possible and already described above.

Taken together, the present disclosure as described above is generally useful in searching for balanced chromosomal aberrations. The disclosure is useful in a methodology that captured sequences are at least at some point also differing from a known contiguous genomic region. According to the instant disclosure, desired target sequences may capture a specific nucleic acid molecule. However, in the case of a balanced chromosomal aberration this captured nucleic acid molecule may contain sequences mapping to the corresponding capture probe, but also sequences derived from a fusion partner that can be derived from a different part of the genome. As an example, these sequences can be from a different chromosome (e.g., as a result of a translocation such as t(9;11)(p22;q23))), but also from the same chromosome (e.g., as a result of an inversion such as inv(16)(p13q22)). According to embodiments of the instant disclosure, these chimeric sequences provide a possibility to further identify hitherto unknown fusion partner genes resulting from balanced chromosomal aberrations. Generally, the present disclosure is also directed to the detection, characterization, sub-type classification and/or optimal treatment of diseases, in particular malignancies like lymphomas or leukemias, especially AML.

Additionally, the present disclosure enables the detection of fusion genes, point mutations, as well as deletions and insertions in a one-step approach. Such genetic characterization enables the detection and/or an optimal treatment of diseases, in particular malignancies like lymphomas or leukemias, especially AML.

Further, the present disclosure is also directed to the detection of at least one further mutation, such as at least one further deletion, for example a deletion in the breakpoint area of the translocation or inversion, at least one further insertion and/or at least one further substitution in the genome (e.g., at least one single nucleotide polymorphism (SNP)).

The following examples, sequence listing, and figures are provided for the purpose of demonstrating various embodiments of the instant disclosure and aiding in an understanding of the present disclosure, the true scope of which is set forth in the appended claims. These examples are not intended to, and should not be understood as, limiting the scope or spirit of the instant disclosure in any way. It should also be understood that modifications can be made in the procedures set forth without departing from the spirit of the disclosure.

### EXAMPLES

### Example 1

This example describes, generally, how to perform selection that allows for rapid and efficient characterization of balanced chromosomal aberrations such as translocations or inversions, occurring in particular in cancer genomes. Moreover, this example describes the method of how to discover a hitherto unknown fusion partner gene. Microarrays having immobilized probes are used in one- or multiple rounds of hybridization selection with a target of total genomic DNA, and the selected sequences are amplified by LM-PCR. Microarray laboratory steps are principally based on the NimbleGen User Guide Version 3.1, 7 Jul. (2009).

A.) Preparation of the Genomic DNA and Double-Stranded Linkers.

DNA is fragmented using nebulization to an average size of ˜500 base pairs. A reaction to polish the ends of the nebulized DNA fragments is set up according to the following:

Polishing Master Mix

| | |
|---|---|
| 10X NEB T4 DNA Polymerase Buffer (NEB2) | 12 μl |
| water | 9 μl |
| 100X NEB BSA | 1 μl |
| 25 mM dNTP stock (mixing 100 μl each of 100 mM dA, dC, dT, | 5 μl |
| and dGTP) | |
| 100 mM ATP (ribonucleotide) | 1 μl |
| 3 U/μl T4 DNA Polymerase | 6 μl |
| 10 U/μl T4 Polynucleotide Kinase | 6 μl |
| Total | 40 μl |

The reaction is incubated at 20 minutes at 12° C., 20 minutes at 25° C. and 20 minutes at 75° C. The reaction is then subjected to linker ligation. Two complementary oligonucleotides are annealed to create a double-stranded linker, by mixing the following:

gSel3=
5′-CTC GAG AAT TCT GGA TCC TC-3′
gSel4-Pi=
5′-Phos/GAG GAT CCA GAA TTC TCG AGT T-3′

In 0.2 ml strip tubes, 5 μl of 4,000 μM gSel3 with 5 μl of 4,000 μM gSel4-Pi are mixed. As many tubes as possible are prepared with the synthesized primers. Each tube now contains 2,000 μM of each linker.

The PCR reaction is performed as follows: 95° C. for 5 minutes, ramp cool 0.1° C. per second to 12° C., and hold at 12° C. Using Oligo Annealing Buffer (OAB) a solution is created containing 500 μM working stock of linkers.

The Oligo Annealing Buffer (OAB) comprises:

| | | |
|---|---|---|
| 1M Tris-HCl (pH 7.8) | 100 | μl |
| 0.5M EDTA (pH 8.0) | 20 | μl |
| 5M NaCl | 100 | μl |
| VWR water | 9.78 | ml |
| Total | 10 | ml |

According to the exemplified embodiment, the length of the 2 complementary oligonucleotides 1 and 2 is between 12 and 24 nucleotides, and the sequence is selected depending upon the functionality desired.

B.) Ligation of Linkers to Genomic DNA Fragments.

The following reaction to ligate the linkers to genomic DNA fragments is set up: to 5 μg of polished DNA, 8 μl of 500 μM annealed linker stock are added:

Ligation Master Mix

| | |
|---|---|
| 10X NEB Buffer 2 | 8 μl |
| 100 mM ATP (ribonucleotide) | 4 μl |
| VWR water | 51 μl |
| T4 DNA Ligase | 10 μl |
| Total | 73 μl |

The ligation reaction is incubated in a thermocycler at 25° C. for 90 minutes. Ligated genomic DNA is subsequently purified in order to remove small fragments.

C.) Primary Selection and Capture of Hybrids.

To prepare the genomic DNA sample for hybridization to the microarray, linker modified genomic DNA (5 μg) is resuspended in 4.8 μl of nuclease-free water and combined with 8.0 μl NimbleGen Hybridization Buffer (Roche NimbleGen, Inc., Madison, Wis.), 3.2 μl Hybridization Additive (Roche NimbleGen, Inc), in a final volume of 164 The samples are heat-denatured at 95° C. for 5 minutes and transferred to a 42° C. heat block.

To capture the target genomic DNA on the microarray, samples are hybridized to NimbleGen CGH arrays, manufactured as described in U.S. Pat. No. 6,375,903 (Roche NimbleGen, Inc.). Maskless fabrication of capture oligonucleotides on the microarrays is performed by light-directed oligonucleotide synthesis using a digital micromirror as described in Singh-Gasson et al. (1999, Nat. Biotech. 17:974-978) as performed by a maskless array synthesizer. Gene expression analysis using oligonucleotide arrays produced by maskless photolithography is described in Nuwaysir et al. (2002, Genome Res. 12:1749-1755). Hybridization is performed in a MAUI Hybridization System (BioMicro Systems, Inc., Salt Lake City, Utah) according to manufacturer instructions for 72 hours at 42° C. using mix mode B. Following hybridization, arrays are washed twice with Wash Buffer I (0.2×SSC, 0.2% (v/v) SDS, 0.1 mM DTT, NimbleGen Systems) for a total of 2.5 minutes. Arrays are then washed for 1 minute in Wash Buffer II (0.2×SSC, 0.1 mM DTT, NimbleGen Systems) followed by a 15 second wash in Wash Buffer III (0.05×SSC, 0.1 mM DTT, Roche NimbleGen, Inc.). To elute the genomic DNA hybridized to the microarray, the arrays are washed with 425 μl of the 125 mM NaOH solution using an elution chamber. The eluted DNA then is purified (Qiagen MinElute column protocol).

D.) Amplification of the Primary Selected DNA.

The primary selected genomic DNA is amplified as described below. Twelve separate replicate amplification reactions are set up. Only one oligonucleotide primer is required because each fragment has the same linker ligated to each end:

Reaction reagents: final total volume of 50 μl

Template: primary selection material 25 μl

LM-PCR Master Mix Amount

| | |
|---|---|
| 10X ThermoPol Reaction Buffer | 4.9 μl |
| 25 mM dNTP | 0.5 μl |
| 40 μM gSel3 | 6.25 μl |
| VWR water | 11.35 μl |
| 5 U/μl Taq DNA Polymerase | 1 μl |
| 0.05 U/μl PfuTurbo DNA Polymerase | 1 μl |
| Total | 25 μl |

The reactions are amplified according to the following program:

| Cycle number | Denaturation | Annealing | Polymerization |
|---|---|---|---|
| 1 | 2 min at 95° C. | | |
| 2-28 | 1 min at 95° C. | 1 min at 60° C. | 2 min at 72° C. |
| 1 | | | 5 min at 72° C. |

The amplification products are purified using a QIAquick PCR purification kit. The eluted samples are pooled and the concentration of amplified primary selected DNA is determined by spectrophotometry.

### Example 2

### Detection of Balanced Chromosomal Aberrations of AML

DNA sequence enrichment from complex genomic samples using microarrays and a 454 PicoTiterPlate (PTP) pyrosequencing assay with long-oligonucleotide sequence capture arrays was applied to allow a comprehensive genetic characterization in a one-step procedure. Three AML cases were analyzed with either known chromosomal aberrations—inversions and translocations—leading to fusion genes (CBFB-MYH11, MLL-MLLT3, MLL-unidentified fusion partner) according to the experimental conditions explained in the generic example (Example 1).

A high-density oligonucleotide microarray that captured short segments that correspond to 92 individual gene exon regions (approximately 1.91 Mb of total sequence, sequence build HG18) was synthesized according to standard Roche NimbleGen, Inc (385K format; Madison, Wis.). microarray manufacturing protocols. Overlapping microarray probes of more than 60 bases each on the array spanned each target genome region, with a probe positioned each 10 bases for the forward strand of the genome. In addition, full genomic regions were represented for three additional target genes (MLL, RUNX1, CBFB).

To test the performance of the capture system, the genomic design was first used to capture fragmented genomic DNA from an acute myeloid leukemia (AML) patient sample (case N1). This case harboured an inv(16)(p13q22) aberration, as confirmed by cytogenetics. On a molecular level the CBFB gene is fused to MYH11 on chromosome 16. A second case (N3) was characterized by a translocation with a confirmed known partner gene (fusion between MLL and MLLT3). This patient harboured a translocation t(9;11)(p22;q23). A third case was analyzed that was known to have a rearrangement of chromosomal material with involvement of the cytoband 11 q23. However, neither cytogenetic nor molecular genetic analyses were able to fully identify the balanced chromosomal aberration or to fully resolve a molecular fusion gene. This genomic region is frequently involved in rearrangements. The MLL gene is known to have many partner genes. The majority of partner genes is known but several genomic loci are not yet fully characterized (Meyer C et al., Leukemia. 2009 August; 23(8):1490-9.). As such, this case N5 can be characterized as MLL-X where the unknown partner gene was suspected to be located on chromomal band 19p13.1.

Case N1: AML with inv(16)(p13q22) and molecular fusion of CBFB-MYH11

Case N3: AML with t(9;11)(p22;q23) and molecular fusion of MLL-MLLT3

Case N5: AML with t(11q23)/MLL and unknown partner gene fused to MLL

Briefly, genomic DNA (20 μg) was subjected to nebulization. 5 μg of the fragmented DNA was processed according to the standard NimbleGen laboratory workflow, i.e. polishing, linker ligation. The linker-terminated fragments were denatured to produce single stranded products that were exposed to the capture microarrays under hybridization conditions in the presence of 1× hybridization buffer (Roche NimbleGen, Inc.) for approximately 72 hours at 42° C. with active mixing using a MAUI hybridization station (Roche NimbleGen, Inc.). Single-stranded molecules that did not hybridize were washed from the microarrays under stringent washing conditions, 3×5 minutes with Stringent Wash Buffer (Roche NimbleGen, Inc.) and rinsed with Wash Buffers I, II, and III (Roche NimbleGen, Inc.). Fragments captured on the microarrays were immediately eluted with 125 mM NaOH and processed for amplification by LM-PCR using a primer complementary to the previously ligated linker oligonucleotides.

To quantify enrichment of the sample genomic DNA, four regions were selected for quantitative PCR (qPCR). These regions were amplified using the following primers (Primer Sequences (5′→3′). These assays act as a proxy for estimating the enrichment of larger populations of capture targets without a need for sequencing. If qPCR analysis using NSC assays indicates a successful capture of the control loci, it is likely that the experimental loci of interest were also successfully captured.

NSC-0237
F:
(SEQ ID NO: 3)
CGCATTCCTCATCCCAGTATG
R:
(SEQ ID NO: 4)
AAAGGACTTGGTGCAGAGTTCAG
NSC-0247
F:
(SEQ ID NO: 5)
CCCACCGCCTTCGACAT
R:
(SEQ ID NO: 6)
CCTGCTTACTGTGGGCTCTTG
NSC-0268
F:
(SEQ ID NO: 7)
CTCGCTTAACCAGACTCATCTACTGT
R:
(SEQ ID NO: 8)
ACTTGGCTCAGCTGTATGAAGGT
NSC-0272
F:
(SEQ ID NO: 9)
CAGCCCCAGCTCAGGTACAG
R:
(SEQ ID NO: 10)
ATGATGCGAGTGCTGATGATG

After a single round of microarray capture, the enriched and LM-PCR amplified samples were compared against the non-enriched and LM-PCR amplified samples (i.e. not hybridized to a capture array) using a LighCyclerLC480 real-time PCR system (Roche Applied Science, Mannheim, Germany) measuring SYBR green fluorescence according to manufacturer's protocols. In detail, 218-fold (case N1), 172-fold (case N3), and 281-fold (case N5) enrichment was achieved for the three AML samples. The theoretical maximum enrichment level was 600 fold (3,000 Mb in the genome and 5 Mb of total sequence).

Samples eluted from the capture microarrays were ligated to 454-sequencing-compatible linkers, amplified using emulsion PCR on beads and sequenced using the 454 FLX sequencing instrument Titanium chemistry workflow (454, Branford Conn.). DNA sequencing of the three samples on the 454 FLX instrument generated 84.0 Mb (case N1), 54.8 Mb (case N3), and 65.8 Mb of total sequence (case N5), respectively. Individual reads were as follows: case N1: 252,651 sequencing reads, case N3: 167,233 reads, case N5: 211,114 reads, respectively.

Following in silico removal of the linker sequence (e.g. with the gsMapper Version 2.0.01 from Life Sciences, USA.), each sequencing read was compared to the entire appropriate version of the Human Genome using BLAST analysis (Altschul, et al., 1990, J. Mol. Biol. 215:403-410; incorporated herein by reference in its entirety) using a cut-off score of e=10⁻⁴⁸, tuned to maximize the number of unique hits. Captured sequences that, according to the original BLAST comparison, map uniquely back to regions within the target regions were considered sequencing hits. These were then used to calculate the % of reads that hit target regions, and the fold sequencing coverage for the entire target region. Data was visualized using SignalMap software (Roche NimbleGen, Inc.). BLAST analysis showed that 88.7% (case N1), 89.6% (case N3), and 88.4% (case N5) of reads, respectively, mapped back uniquely to the genome; 61.4% (case N1), 65.5% (case N1), and 80.5% (case N1) were from targeted regions. The median per-base coverage for each sample was 22.8-fold (case N1), 15.9-(case N3) and 24.1-fold coverage (case N3), respectively (Table 1).

TABLE 1

| | | | Percentage of | Percentage of | Median Fold |
|---|---|---|---|---|---|
| | | FLX - | Reads Mapped | Total Reads That | Coverage for |
| | qPCR Fold | Yield | Uniquely to the | Mapped to | Target |
| DNA Sample | Enrichment | (Mb) | Genome | Selection Targets | Regions |
| N1 | 218 | 80.8 | 88.6% | 61.4% | 22.8 |
| N3 | 172 | 53.0 | 89.6% | 65.5% | 15.9 |
| N5 | 281 | 64.0 | 88.4% | 80.5% | 24.1 |

Reads that did not uniquely map back to the reference genome were not discarded, but were analyzed further for the detection of chimeric reads. As such, reads that were “partially” mapped to the reference genome, or reads that were discovered to not map to the genome were further filtered to detect chimeric sequences.

Case N1.

Here a total of 13 reads was observed to carry sequence information that map both to the MYH11 gene and the CBFB gene. For example sequence read No. 1 of Table 1 has a total length of 449 bases. Of these bases 1-240 map to chromosome 16 (Start: 15,722,449; End: 15,722,688). Of these 242 bases, 230 were found to be representative for MYH11. The remainder sequence of the bases 241 to 449 was detected to represent the CBFB gene with chromosome 16 Start at 65,678,383 and End at 65,678,590.

TABLE 2

| | | | | | | | | Gene |
|---|---|---|---|---|---|---|---|---|
| No. | Start | End | Length | Location | Base start | Base end | Identity | Symbol |
| 1 | 1 | 240 | 449 | chr16 | 15,722,449 | 15,722,688 | (230/242 ident) | MYH11 |
| | 449 | 241 | 449 | chr16 | 65,678,383 | 65,678,590 | (205/210 ident) | CBFB |
| 2 | 62 | 492 | 492 | chr16 | 15,722,691 | 15,723,119 | (428/431 ident) | MYH11 |
| | 61 | 20 | 492 | chr16 | 65,678,590 | 65,678,631 | (42/42 ident) | CBFB |
| 3 | 509 | 287 | 509 | chr16 | 15,722,468 | 15,722,690 | (220/225 ident) | MYH11 |
| | 1 | 286 | 509 | chr16 | 65,678,301 | 65,678,588 | (276/289 ident) | CBFB |
| 4 | 181 | 1 | 326 | chr16 | 15,722,692 | 15,722,865 | (169/181 ident) | MYH11 |
| | 182 | 326 | 326 | chr16 | 65,678,589 | 65,678,734 | (142/146 ident) | CBFB |
| 5 | 1 | 233 | 461 | chr16 | 15,722,452 | 15,722,688 | (224/237 ident) | MYH11 |
| | 441 | 234 | 461 | chr16 | 65,678,382 | 65,678,590 | (205/210 ident) | CBFB |
| 6 | 266 | 1 | 463 | chr16 | 15,722,692 | 15,722,952 | (254/266 ident) | MYH11 |
| | 267 | 463 | 463 | chr16 | 65,678,589 | 65,678,783 | (193/197 ident) | CBFB |
| 7 | 356 | 1 | 431 | chr16 | 15,722,692 | 15,723,049 | (347/360 ident) | MYH11 |
| | 357 | 411 | 431 | chr16 | 65,678,589 | 65,678,643 | (55/55 ident) | CBFB |
| 8 | 159 | 475 | 475 | chr16 | 15,722,691 | 15,723,004 | (313/317 ident) | MYH11 |
| | 158 | 20 | 475 | chr16 | 65,678,590 | 65,678,726 | (135/139 ident) | CBFB |
| 9 | 1 | 142 | 502 | chr16 | 15,722,553 | 15,722,688 | (134/142 ident) | MYH11 |
| | 502 | 143 | 502 | chr16 | 65,678,232 | 65,678,590 | (354/362 ident) | CBFB |
| 10 | 198 | 489 | 489 | chr16 | 15,722,691 | 15,722,983 | (291/293 ident) | MYH11 |
| | 197 | 1 | 489 | chr16 | 65,678,590 | 65,678,779 | (186/197 ident) | CBFB |
| 11 | 1 | 241 | 356 | chr16 | 15,722,449 | 15,722,688 | (230/243 ident) | MYH11 |
| | 356 | 242 | 356 | chr16 | 65,678,479 | 65,678,590 | (111/116 ident) | CBFB |
| 12 | 295 | 1 | 516 | chr16 | 15,722,692 | 15,722,984 | (287/297 ident) | MYH11 |
| | 296 | 516 | 516 | chr16 | 65,678,589 | 65,678,811 | (221/223 ident) | CBFB |
| 13 | 333 | 508 | 508 | chr16 | 15,722,691 | 15,722,866 | (176/176 ident) | MYH11 |
| | 332 | 1 | 508 | chr16 | 65,678,590 | 65,678,918 | (323/333 ident) | CBFB |

Case N3.

Here a total of 8 reads was observed to carry sequence information that map both to the MLL gene and the MLLT3 gene. For example sequence read No. 1 of Table 3 has a total length of 436 bases. Of these bases 296-436 map to chromosome 11 (Start: 117859810; End: 117859950). Of these 141 bases, 140 were found to be representative for MLL. The remainder sequence of the bases 1 to 194 was detected to represent the MLLT3 gene with chromosome 11 Start at 20,350,483 and End at 20,350,776.

TABLE 3

| | | | | | | | | Gene |
|---|---|---|---|---|---|---|---|---|
| No. | Start | End | Length | Location | Base start | Base end | Identity | Symbol |
| 1 | 436 | 296 | 436 | chr11 | 117859810 | 117859950 | (140/141 ident) | MLL |
| | 1 | 294 | 436 | chr9 | 20350483 | 20350776 | (294/294 ident) | MLLT3 |
| 2 | 164 | 1 | 401 | chr11 | 117859951 | 117860114 | (164/164 ident) | MLL |
| | 168 | 401 | 401 | chr9 | 20350778 | 20351012 | (233/235 ident) | MLLT3 |
| 3 | 163 | 1 | 278 | chr11 | 117859951 | 117860113 | (163/163 ident) | MLL |
| | 167 | 278 | 278 | chr9 | 20350778 | 20350889 | (112/112 ident) | MLLT3 |
| 4 | 91 | 425 | 425 | chr11 | 117859951 | 117860281 | (330/336 ident) | MLL |
| | 87 | 1 | 425 | chr9 | 20350778 | 20350864 | (87/87 ident) | MLLT3 |
| 5 | 270 | 490 | 490 | chr11 | 117859951 | 117860173 | (220/223 ident) | MLL |
| | 266 | 1 | 490 | chr9 | 20350778 | 20351043 | (266/266 ident) | MLLT3 |
| 6 | 480 | 345 | 480 | chr11 | 117859815 | 117859950 | (135/136 ident) | MLL |
| | 1 | 343 | 480 | chr9 | 20350432 | 20350776 | (343/345 ident) | MLLT3 |
| 7 | 237 | 492 | 492 | chr11 | 117859951 | 117860208 | (255/258 ident) | MLL |
| | 233 | 1 | 492 | chr9 | 20350778 | 20351010 | (233/233 ident) | MLLT3 |
| 8 | 62 | 381 | 381 | chr11 | 117859951 | 117860270 | (318/322 ident) | MLL |
| | 58 | 1 | 381 | chr9 | 20350778 | 20350835 | (58/58 ident) | MLLT3 |

Case N5.

In this case, a translocation t(11;19)(q23;p13) had been observed in chromosome banding analysis and the involvement of the MLL gene had been proven by fluorescence in situ hybridization. However, using RT-PCR no fusion transcripts could be amplified. In contrast, the next-generation sequencing approach identified chimeric reads.

Here a total of 5 reads was observed to carry sequence information that map to the MLL gene. For example sequence read No. 1 of Table 5 has a total length of 480 bases. Of these bases 310-480 map to chromosome 11 (Start: 117860271; End: 117860441). Of these 173 bases, 169 were found to be representative for MLL. The remainder sequence of the bases 1 to 309 was detected to represent the ELL gene with chromosome 19 Start at 18430871 and End at 18431174 (identity was 299/309 bases). Since in this case both cytogenetic analysis and molecular PCR-based assays failed to reveal the partner gene, the present disclosure is a useful method to detect any fusion partner gene. It is sufficient to capture one partner gene, in this case MLL, and to capture and subsequently sequence any occurring chimeric reads.

This is illustrated by additional chimeric reads which were composed of SFRS14 (splicing factor, arginine/serine-rich 14; also located on 19p13 centromeric of ELL) and MLL. This suggested that a deletion had occurred in the breakpoint area and thus prevented the formation of a reciprocal ELL-MLL fusion gene. SNP array analysis (Affymetrix genome-wide human SNP array 6.0) were performed and data from the SNP microarrays demonstrated a 615 kb deletion on 19p13, flanked by ELL and SFRS14, spanning from chr19: 18,346,048-18,961,490. As such, a micro deletion was causative for the fusion of SFRS14 to MLL in the reciprocal setting.

TABLE 4

| | | | | | | | | Gene |
|---|---|---|---|---|---|---|---|---|
| No. | Start | End | Length | Location | Base start | Base end | Identity | Symbol |
| 1 | 480 | 310 | 480 | chr11 | 117860271 | 117860441 | (169/173 ident) | MLL |
| | 1 | 309 | 480 | chr19 | 18430871 | 18431174 | (299/309 ident) | ELL |
| 2 | 414 | 373 | 414 | chr11 | 117860400 | 117860441 | (42/42 ident) | MLL |
| | 1 | 372 | 414 | chr19 | 18430807 | 18431174 | (361/374 ident) | ELL |
| 3 | 123 | 471 | 471 | chr11 | 117860460 | 117860809 | (343/351 ident) | MLL |
| | 122 | 9 | 471 | chr19 | 18962849 | 18962964 | (110/116 ident) | SFRS14 |
| 4 | 395 | 458 | 458 | chr11 | 117860460 | 117860523 | (64/64 ident) | MLL |
| | 394 | 1 | 458 | chr19 | 18962849 | 18963236 | (383/394 ident) | SFRS14 |
| 5 | 123 | 418 | 418 | chr11 | 117860460 | 117860752 | (291/296 ident) | MLL |
| | 122 | 9 | 418 | chr19 | 18962849 | 18962964 | (109/116 ident) | SFRS14 |

These data illustrate the advantages of the present disclosure. A programmable high-density array platform with 385,000 probes was used. The probes were readily able to capture up to 5 Mb of total sequence. In addition, to the specificity of the assay, the high yields of the downstream DNA sequencing steps are consistently superior to the routine average performance using non-captured DNA sources. This is attributed to the capture-enrichment process providing a useful purification of unique sequences away from repeats and other impurities that can confound, for example, the first emulsion PCR step of the 454 sequencing process.

In the present example, a computer program was used to map the obtained reads both exactly against the human genome, but also searched for chimeric sequences mapping to different regions in the genome. By this approach all corresponding fusion genes in our examples were detected as CBFB-MYH11 as well as the reciprocal MYH11-CBFB and MLL-MLLT3 and MLLT3-MLL, respectively. It was further demonstrated that fusion genes can be detected in a one-step methodological approach using the combination of a targeted DNA sequence enrichment assay followed by next-generation sequencing technology. In this embodiment, the genomic representation of only one of the partner genes of a chimeric fusion on this capture platform is sufficient to identify also any potentially unknown partner gene as a result of a balanced chromosomal aberration. Additionally, reciprocal fusion constructs will be revealed. As such, this novel assay has a strong potential to become a valuable method for a comprehensive genetic characterization of particularly leukemias and other malignancies.

All publications, patents and applications are hereby incorporated by reference in their entirety to the same extent as if each such reference was specifically and individually indicated to be incorporated by reference in its entirety.

While this disclosure has been described as having an exemplary design, the present disclosure may be further modified within the spirit and scope of this disclosure. This application is therefore intended to cover any variations, uses, or adaptations of the disclosure using its general principles. Further, this application is intended to cover such departures from the present disclosure as come within the known or customary practice in the art to which this disclosure pertains.

What is claimed is:

1. A method for detecting balanced chromosomal aberrations in a genome of an organism, the method comprising the steps of:
(a) exposing fragmented, denatured nucleic acid molecules of the genome to a plurality of oligonucleotide probes bound to different positions of a solid support, the nucleic acid molecules having an average size of about 100 to about 1000 nucleotide residues and the oligonucleotide probes having an average size of about 20 to about 100 nucleotide residues;
(b) separating nucleic acid molecules bound to one or more of the oligonucleotide probes from nucleic acid molecules not bound to one or more of the oligonucleotide probes;
(c) eluting the nucleic acid molecules bound to one or more of the oligonucleotide probes from the solid support;
(d) sequencing the nucleic acid molecules eluted in said step of eluting, whereby a determined sequence for the nucleic acid molecules is obtained;
(e) comparing the determined sequence to a database comprising a reference genome sequence;
(f) identifying sequences in the determined sequence which only partially match or do not match with sequences of the reference genome; and
(g) detecting at least one balanced chromosomal aberration.

2. The method according to claim 1, wherein the oligonucleotide probes comprise a linker for binding to the solid support.

3. The method according to claim 1, further comprising the step of ligating at least one adaptor molecule to at least one end of the nucleic acid molecules prior to step (a).

4. The method according to claim 3, further comprising the step of amplifying the nucleic acid molecules which bound to one or more of the oligonucleotide probes with at least one primer comprising a sequence which specifically hybridizes to the adaptor molecule, said step of amplifying being carried out after step (c).

5. The method according to claim 4, wherein the at least one primer and the at least one adaptor sequence are removed in silico prior to step (d)

6. The method according to claim 1, further comprising the step of purifying the nucleic acid molecules which bound to one or more of the oligonucleotide probes prior to step (d).

7. The method according to claim 6, further comprising the step of amplifying the purified nucleic acid molecule prior to step (d) by emulsion polymerase chain reaction.

8. The method according to claim 1, wherein the nucleic acid molecules are genomic DNA molecules containing at least one chromosome of an organism with a size of at least about 50 kb.

9. The method according to claim 7, wherein the oligonucleotide probes contain at least one of an exon sequence, an intron sequence, and a regulatory sequence from the at least one chromosome of the organism.

10. The method according to claim 7, wherein probes with highly repetitive sequences are excluded.

11. The method according to claim 7, wherein the database comprising the reference genome contains at least 95% of the at least one chromosome of the organism.

12. The method according to claim 1, wherein the solid support comprises one of a nucleic acid microarray and a population of beads.

13. A method for detecting balanced chromosomal aberrations in a genome, the method comprising the steps of:
(a) providing a solid support comprising a plurality of different oligonucleotide probes bound to different positions of the solid support, the oligonucleotide probes having an average size of about 20 to about 100 nucleotides;
(b) providing a plurality of fragmented and denatured nucleic acid molecules having an average size of about 100 to about 1000 nucleotide residues;
(c) amplifying the oligonucleotide probes, whereby a plurality of amplification products are generated, the plurality of amplification products including a binding moiety, the amplification products being maintained in solution;
(d) hybridizing the target nucleic acid molecules to the amplification products in solution under specific hybridizing conditions, whereby a plurality of hybridization complexes are generated;
(e) separating the hybridization complexes from nucleic acid molecules not hybridized to the amplification products;
(f) separating the hybridized target nucleic acid molecules from the amplification product comprising the hybridization complex,
(g) sequencing the target nucleic acid molecules separated in said step of separating, whereby a determined sequence for the nucleic acid molecules is obtained;
(h) comparing the determined sequence to a database comprising a reference genome;
(i) identifying sequences in the determined sequence which only partially match or do not match with sequences of the reference genome,
(i) detecting at least one balanced chromosomal aberration.

14. The method according to claim 13, wherein the oligonucleotide probes comprise a linker for binding to the solid support.

15. The method according to claim 13, wherein the oligonucleotide probes comprise a primer binding sequence at at least one end.

16. The method according to claim 13, wherein the binding moiety is a biotin binding moiety.

17. The method according to claim 13, wherein said step of separating comprises binding said biotin binding moiety to a streptavidin coated substrate.

18. The method according to claim 13, wherein the nucleic acid molecules are genomic DNA molecules containing at least one chromosome of an organism with a size of at least about 50 kb.

19. The method according to claim 18, wherein the oligonucleotide probes contain at least one of an exon sequence, an intron sequence, and a regulatory sequence from the at least one chromosome of the organism.

20. The method according to claim 19, wherein probes with highly repetitive sequences are excluded.


SEQUENCE LISTING (10 sequences)

SEQ ID NO: 1 - DNA, 20 bp, Artificial Sequence
  probe
ctcgagaattctggatcctc

SEQ ID NO: 2 - DNA, 22 bp, Artificial Sequence
  probe
gaggatccagaattctcgagtt

SEQ ID NO: 3 - DNA, 21 bp, Artificial Sequence
  primer
cgcattcctcatcccagtatg

SEQ ID NO: 4 - DNA, 23 bp, Artificial Sequence
  reverse primer
aaaggacttggtgcagagttcag

SEQ ID NO: 5 - DNA, 17 bp, Artificial Sequence
  forward primer
cccaccgccttcgacat

SEQ ID NO: 6 - DNA, 21 bp, Artificial Sequence
  reverse primer
cctgcttactgtgggctcttg

SEQ ID NO: 7 - DNA, 26 bp, Artificial Sequence
  forward primer
ctcgcttaaccagactcatctactgt

SEQ ID NO: 8 - DNA, 23 bp, Artificial Sequence
  reverse primer
acttggctcagctgtatgaaggt

SEQ ID NO: 9 - DNA, 20 bp, Artificial Sequence
  forward primer
cagccccagctcaggtacag

SEQ ID NO: 10 - DNA, 21 bp, Artificial Sequence
  reverse primer
atgatgcgagtgctgatgatg